(19) United States
(12) Patent Application Publication
(10) Pub. No.: US 2024/0218459 A1
(43) Pub. Date: Jul. 4, 2024

(54) MOLECULAR MARKERS OF PHOTO-SENSITIVITY MODULE RELATED TO RICE BLAST RESISTANCE AND THEIR APPLICATIONS

(71) Applicant: INSTITUTE OF CROP SCIENCES, CHINESE ACADEMY OF AGRICULTURAL SCIENCES, Beijing (CN)

(72) Inventors: Tianqing ZHENG, Beijing (CN); Yuanbao LEI, Beijing (CN); Jianlong XU, Beijing (CN); Ziqi ZHOU, Beijing (CN); Linyun XU, Beijing (CN); Dechen YANG, Beijing (CN); Xiuyun LIN, Beijing (CN); Faruquee MUHIUDDIN, Beijing (CN); Xiuqin ZHAO, Beijing (CN)

(21) Appl. No.: 18/349,935

(22) Filed: Jul. 10, 2023

(30) Foreign Application Priority Data
Dec. 30, 2022 (CN) 202211706892.8

(51) Int. Cl. C12Q 1/6895; C12Q 1/6853

(52) U.S. Cl. CPC C12Q 1/6895; C12Q 1/6853; C12Q 2600/13

(57) ABSTRACT

This invention describes a work including both genetic analysis and development of a marker set for a photo-sensitivity module underlying transgressive photo-sensitivity (TPS) phenomenon found in the breeding improvement of rice blast resistance by the introduction of Pigm gene, belonging to the field of rice molecular genetics and biotechnology breeding. By using bi-parental RIL populations derived from TPS crosses with a Pigm donor as one parent, genetic analysis has been carried out to map the photo-sensitivity module [qHd6+qHd7] under both long-day (LD) and short-day (SD) conditions. With the aid of the molecular marker set proposed by this invention for this module, breeders can predict the degree of TPS in northern early Geng/japonica rice progenies derived from crosses with Pigm donor as one parent when they are doing parental selection or screening progenies carrying Pigm without strong TPS in blast-resistant biotechnology breeding schemes.

This application claims priority to Chinese Patent Application No. 202211706892.8, filed on Dec. 30, 2022, which is incorporated by reference for all purposes as if fully set forth herein.

A Sequence Listing XML file named “10059_0005.xml” created on Sep. 12, 2023, and having a size of 4,542 bytes, is filed concurrently with the specification. The sequence listing contained in the XML file is part of the specification and is herein incorporated by reference in its entirety.

### TECHNICAL FIELD

The present invention belongs to the field of biological breeding and molecular genetics, specifically relates to molecular markers of photo-sensitivity module related to rice blast resistance and their applications. It is suitable for the improvement of rice disease resistance biological breeding, and through the molecular marker identification of the photo-sensitivity module, the photosensitivity of the offspring of rice resistance improvement can be predicted, so as to guide the selection of parents for rice blast resistance improvement.

### BACKGROUND

Pigm is a broad-spectrum blast resistance gene, which is widely used in rice disease resistance improvement (Deng Y, Zhai K, Xie Z, Yang D, Zhu X, Liu J, Wang X, Qin P, Yang Y, Zhang G, Li Q, Zhang J, Wu S, Milazzo J, Mao B, Wang E, Xie H, Tharreau D, He Z (2017). Epigenetic regulation of antagonistic receptors confers rice blast resistance with yield balance. Science 355 (6328):962-965. doi:10.1126/science.aai8898). In breeding practice, our research group found that with the introduction of Pigm gene into northern early Geng/japonica rice, the progenies would show TPS, that is, the photo-sensitivity of the progenies is stronger than either parent (FIG. 1 and FIG. 2). One previous study has reported that the introducing of Pi-2 which is allelic to Pigm gene may cause a slight delay in heading date of rice, but no strong TPS has been found. Genetic mechanism of TPS caused by the introduction of Pigm into northern early Geng/japonica rice has yet to be resolved.

Heading date (time of flowering) is an important agronomic trait, which is critical for regional adaptability of rice cultivars. Rice is a short-day (SD) crop. Photo-sensitivity refers to the response of its growth period to the change of day length, that is, the SD condition promotes flowering, and the long-day (LD) condition delays flowering. It has been known that the heading date of rice is mainly controlled by OsGI-Hd1-Hd3a/RFT1 and Ghd7-Ehd1-Hd3a/RFT1 regulating pathways (Zhou S, Zhu S, Cui S, Hou H, Wu H, Hao B, Cai L, Xu Z, Liu L, Jiang L, Wang H, Wan J (2021). Transcriptional and post-transcriptional regulation of heading date in rice. New Phytol 230 (3):943-956. doi:10.1111/nph.17158). Among them, Ghd7 encodes a protein with CCT domain, and delays heading by interacting with key genes such as Hd1 and DTH8 under LD conditions and inhibiting downstream Ehd1 gene (Zong W, Ren D, Huang M, Sun K, Feng J, Zhao J, Xiao D, Xie W, Liu S, Zhang H, Qiu R, Tang W, Yang R, Chen H, Xie X, Chen L, Liu Y G, Guo J (2021). Strong photoperiod sensitivity is controlled by cooperation and competition among Hd1, Ghd7, and DTH8 in rice heading. New Phytol 229 (3):1635-1649. doi:10.1111/nph.16946). In northern Geng/japonica rice, Ghd7 has a larger impact on maturity time and grain yield. Previous researchers have developed a series of molecular markers for Ghd7, however, few of them are fit for breeding applications. Among them, RM5436 is a marker that is a bit far away from Ghd7, and crossover is prone to happen between this marker and the target locus. Se9151 and Se9153 belong to restricted endonuclease markers (Xue W, Xing Y, Weng X, Zhao Y, Tang W, Wang L, Zhou H, Yu S, Xu C, Li X, Zhang Q (2008). Natural variation in Ghd7 is an important regulator of heading date and yield potential in rice. Nat Genet 40 (6):761-767. doi:10.1038/ng.143). The testing cost is relatively high, and there is a basic requirement of testing conditions for this marker. In addition, the existing molecular markers of photo-sensitive genes aim at a single locus. The molecular evaluation of more than two related loci, namely module, especially with molecular markers that can effectively predict the TPS of Pigm gene introduced progenies, has not yet been provided.

### SUMMARY OF THE INVENTION

In view of the above research background, this invention firstly adopted RIL populations derived from Pigm gene-related TPS cross to carry out genetic analysis and map the photo-sensitivity module [qHd6+qHd7]. On this basis, a set of molecular markers for this module was designed, including one marker M80410 for qHd6 and a marker named ZLM7-1 for qHd7. This marker set is PCR based marker and suitable for the breeding application. The efficiency of the marker set [M80410+ZLM7-1] was firstly validated with the progenies of recombinant inbred lines (RIL) population derived from the above Pigm donor-related TPS cross. Secondly, 120 randomly selected breeding materials, including 70 Heilongjiang and 50 Jilin breeding materials, were genotyped with the marker set. From these 120 materials, 35 randomly selected materials, including 16 Heilongjiang and 19 Jilin breeding materials, were sent to testing cross-making with a pair of near-isogenic lines for Pigm in early Geng/japonica background of Kongyu 131 (KY131) and Kongyu 131 (KY131) is a wide-adapted Heilongjiang cultivar. The results showed that the marker set, [M80410+ZLM7-1], can effectively identify transgressive photo-sensitivity (TPS) in Pigm-related early Geng/japonica breeding scheme. The average accuracy of prediction is about 91.4%, including 81.2% for Heilongjiang breeding materials and 100% for Jilin breeding materials. Hereby, the invention is accomplished.

The invention first provides a molecular marker set, [M80410+ZLM7-1], for blast-resistant gene Pigm-related photo-sensitivity module, [qHd6+qHd7].

Further, the present invention provides blast-resistance Pigm related photo-sensitivity module, [qHd6+qHd7], which contains two loci. One is in the 8,665,233-9,600,319 bp interval of rice chromosome 6, named qHd6, and the other is located in the 8,556,052-11,072,552 bp interval of rice chromosome 7, named qHd7.

Specifically, genotypes of the two loci, qHd6 and qHd7, can be identified by PCR-based primers.

Preferably, the PCR primer for the locus qHd6, is M80410 with forward primer sequence: GGATTGTCTTGTCTCTCTCGC (SEQ ID NO: 3) and the reverse primer sequence: CAGGACTTAGGGTTTCTCTCTTT (SEQ ID NO: 4); the PCR primer pair of the locus qHd7 is the molecular marker ZLM7-1 with forward primer sequence: TCCCCCAAACATTTTCAGAACAC (SEQ ID NO: 1), and reverse primer sequence: TAGGTGCAGTTGCAGTAGGT (SEQ ID NO: 2).

The invention provides a method for predicting the TPS in northern early Geng/japonica rice progenies from Pigm donor-involved crosses based on a molecular marker set for photo-sensitivity module, [qHd6+qHd7]. It is characterized in that when the genotype for locus qHd6 was identified as A_ or aa (where A_ represents AA or Aa, because this marker is a dominant marker, it is impossible to distinguish the two genotypes which have the same effects). The genotype for locus qHd7 was identified as BB, bb, or Bb. When the genotype of the marker set for the photo-sensitivity module in the rice genome to be tested is A_B_. The TPS is the strongest, and the photo-sensitivity of other genotypes is relatively weak.

Specifically, when M80410 marker primer is used to amplify the rice genome template to be tested by PCR, if about 500 bp bands are amplified, the detected genotype is A_; when the ZLM7-1 marker primer is used to amplify the rice genome template by PCR, if 223 bp bands are amplified, the detection genotype is BB; if about 202 bp bands are amplified, the detection genotype is bb. Through the above detection, if an early Geng/japonica rice with genotype A_B_, it is highly possible to show the strongest TPS.

Further preferably, agarose gel electrophoresis or polyacrylamide gel electrophoresis is used to detect the PCR products, preferably agarose gel electrophoresis is used for the PCR amplification products of M80410 marker primers, and polyacrylamide gel electrophoresis is used for the PCR amplification products of ZLM7-1.

The invention's molecular marker [M80410+ZLM7-1] is adopted jointly in testing the progenies from a population derived from a cross with TPS represented by TH886/XQ62. Progenies with marker genotype of A_B_ presented the strongest TPS. The photo-sensitivity of other genotypes is relatively weak (FIG. 9). When the molecular marker used for the [qHd6+qHd7] photo-sensitivity module is used to predict the early Geng/japonica background material carrying Pigm, such as XQ62 participating in the combination, the average effectiveness can reach 88.6%, and the efficiency in Jilin breeding materials can even reach 100% (Table 1).

Therefore, the molecular marker set of the [qHd6+qHd7] photo-sensitivity module of this invention can be used to improve blast-resistant biological breeding by using Pigm gene. Through the genotypic identification of the molecular marker of the module, it can predict the TPS of the northern early Geng/japonica rice progenies derived from crosses with Pigm as donor parent.

This invention could largely help guide the selection of blast-resistant biotechnological breeding parents and carry out the marker-assisted selection of weak TPS breeding progeny with Pigm gene.

Specifically, with the molecular marker set, breeders can predict the degree of TPS in progenies from the improvement of blast resistance in rice and carry out parental selection.

By using the molecular marker set of the photo-sensitive module [qHd6+qHd7] of the present invention, the specific example is the combination of [M80410+ZLM7-1]. One usage of the marker set is, breeders can get useful progenies with Pigm gene but no strong TPS; another usage is breeders can predict the degree of TPS in F1 derived from crosses with Pigm donor as one parent by testing the marker genotype of [M80410+ZLM7-1] in parents. The joint work of the marker set of [M80410+ZLM7-1] can speed up the breeding process by screening progenies with Pigm but weak TPS.

Furthermore, ZLM7-1 is closely linked to qHd7 and is less prone to be recombinated, making it effective in rice molecular marker assisted breeding. The molecular marker ZLM7-1 of the present invention can be used alone to pre identify the qHd7 genotype of parents, and thus select suitable breeding parents in crossing with donors carrying Pigm genes.

### DESCRIPTION OF THE DRAWINGS

FIG. 1 Transgressive photo-sensitivity (TPS) performance of F₁ hybrids between Jilin breeding material TH886 and Pigm near-isogenic lines XQ62 and Kongyu 131 in Heilongjiang early Geng/japonica background under long-day (LD) conditions.

FIG. 2 Transgressive photo-sensitivity (TPS) performance of F₁ hybrids of Jilin breeding material TH899 and Pigm near-isogenic lines XQ62 and Kongyu 131 in Heilongjiang early Geng/japonica background under long-day (LD) conditions.

FIG. 3 The mapping results of heading date with a set of RIL population derived from the cross between TH886 and XQ62 under long-day (LD) condition in Beijing.

FIG. 4 The mapping results of heading date with a set of RIL population derived from the cross between TH899 and XQ62 under long-day (LD) condition in Beijing.

FIG. 5 The mapping results of heading date with a set of RIL population derived from the cross between TH886 and XQ62 under short-day (SD) condition in Hainan.

FIG. 6 The mapping results of heading date with a set of RIL population derived from the cross between TH899 and XQ62 under short-day (SD) condition in Hainan.

FIG. 7 Genotype of photo-sensitivity module [qHd6+qHd7] with molecular marker M80410, of which about 500 bp bands are blast-resistant with TPS genotype A_.

FIG. 8 Genotype of photo-sensitivity module [qHd6+qHd7] with molecular marker ZLM7-1, in which about 223 bp bands are photo-sensitive genotype BB, and about 202 bp bands are non-sensitive genotype bb.

FIG. 9 The phenotypic values of days from seeding to heading (DTH) of RIL population progenies derived from TH886/XQ62 cross with different genotypes of [qHd6+qHd7] photo-sensitivity module at heading stage under long-day (LD) condition of Beijing.

FIG. 10 The phenotype of a typical cross at heading stage under long-day (LD) condition predicted by the marker set for photo-sensitivity module, [qHd6+qHd7]. Among them, (1) is one breeding parent, (2) F₁ of a cross between breeding parent and XQ62, (3) XQ62 (with Pigm in Kongyu 131 background), (4) F₁ from cross between breeding parent and Kongyu 131, (5) Kongyu 131 (without Pigm).

### SPECIFIC IMPLEMENTATION PLAN

The invention is further described in the following with specific implementation examples. The methods adopted are conventional methods unless otherwise specified.

1. Mapping of qHd7 and qHd6, and Verification with Breeding Materials

### 1.1 Selection of Testing Materials

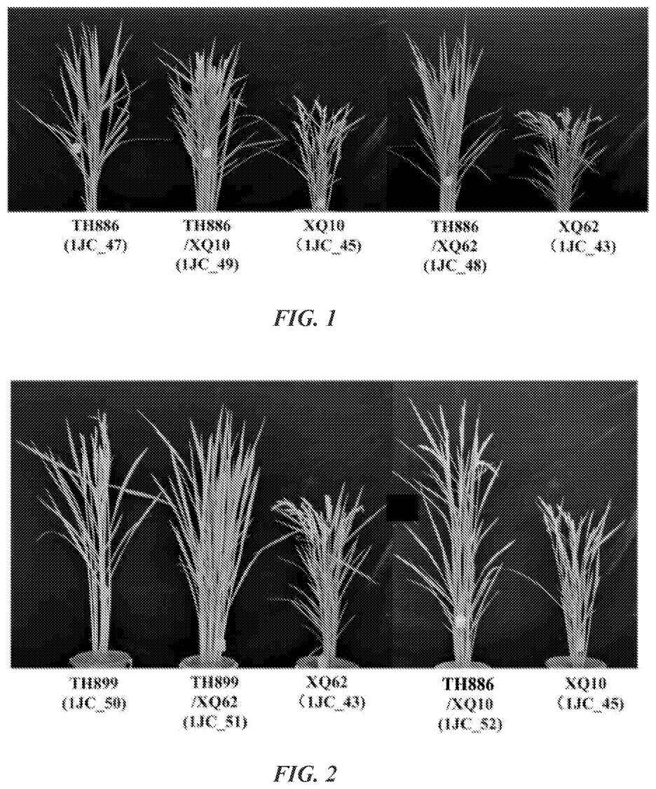

As shown in FIGS. 1 and 2, the initial purpose is to combine the early-maturing background Pigm from Heilongjiang with the high-quality and high-yield Jilin materials TH886 and TH899 for breeding improvement. During the matching process, we found that after the two Jilin materials were paired with XQ62 (Heilongjiang breeding materials background with Pigm gene), F₁ generation had a significant photo-sensitivity phenomenon, while the two materials were paired with Kongyu 131 (Heilongjiang prematurity background without Pigm gene), there was no photo-sensitivity phenomenon.

Therefore, using Jilin materials TH886 and TH899 to prepare hybrid combinations with Heilongjiang early Geng/japonica background Pigm material XQ62, respectively, two RIL populations were derived through single-grain transmission, of which TH886/XQ62 contains 226 F₇ families, and TH899/XQ62 contains 231 F₇ families. 70 Heilongjiang and 50 Jilin breeding materials (35 of them were listed in Table 1) were randomly picked as the validation materials for the markerset.

### 1.2 QTL Mapping

Genomic DNA of RIL population was collected, and genotypic data were obtained by 40K liquid chip. The days from seeding to heading (heading date) of testing materials were collected under long-day (LD) and/or short-day (SD) conditions (the LD condition adopted in this experiment is the natural LD condition in Beijing, and the SD refers to the natural SD condition in Sanya during winter season) and taken as phenotypic values. Genetic mapping analysis was performed using ICIMapping V3.2 package, and the LOD threshold was set to 5.0 in order to map major locus with large effects.

### 1.3 Haplo Type Analysis of Parental Candidate Genes

The haplotype analysis was carried out for the candidate genes located in the interval of the two major QTLs. The CDS sequences of qHd6 and qHd7 candidate genes of four parents of TH886, TH899, Kongyu 131, and XQ62 were analyzed.

### 1.4 Prediction in Testing Cross

The two near-isogenic lines XQ62 and Kongyu 131 were used to cross with 35 materials (16 Heilongjiang and 19 Jilin breeding materials) randomly selected from 120 breeding materials respectively (Table 1), and the performance of F₁ was observed under LD and SD conditions.

TABLE 1
Prediction effect of the molecular marker set, [M80410 + ZLM7-1],
for the transgressive photo-sensitivity (TPS) module, [qHd6 + qHd7],
in crosses between the 35 randomly selected breeding materials and XQ62,
a donor for Pigm in early Geng/japonica background of Kongyu 131 (KY131).

| Female | Male | | | Genotype | Predicted | Real | |
|---|---|---|---|---|---|---|---|
| parent | Parent | Cross | Source ^{a} | of F₁ | TPS ^{b} | TPS | Fit ^{c} |
| ZNG515 | XQ62 | ZNG515/XQ62 | HLJ | A_B_{—} | S | W | N |
| GF16 | XQ62 | GF16/XQ62 | HLJ | A_bb | W | W | Y |
| QP_52 | XQ62 | QP_52/XQ62 | HLJ | A_bb | W | W | Y |
| LH9 | XQ62 | LH9/XQ62 | HLJ | A_bb | W | W | Y |
| LY3252 | XQ62 | LY3252/XQ62 | HLJ | A_B_{—} | S | S | Y |
| ZNG9 | XQ62 | ZNG9/XQ62 | HLJ | A_bb | W | W | Y |
| LH00_233 | XQ62 | LH00_233/XQ62 | HLJ | A_bb | W | W | Y |
| LJ04_109 | XQ62 | LJ04_109/XQ62 | HLJ | A_B_{—} | S | S | Y |
| LG20 | XQ62 | LG20/XQ62 | HLJ | A_bb | W | W | Y |
| LG1755 | XQ62 | LG1755/XQ62 | HLJ | A_bb | W | S | N |
| LN2 | XQ62 | LN2/XQ62 | HLJ | A_bb | W | S | N |
| PN8 | XQ62 | PN8/XQ62 | HLJ | A_B_{—} | S | W | N |
| LG29 | XQ62 | LG29/XQ62 | HLJ | A_B_{—} | S | S | Y |
| LG57 | XQ62 | LG57/XQ62 | HLJ | A_bb | W | W | Y |
| N293 | XQ62 | N293/XQ62 | HLJ | A_B_{—} | S | S | Y |
| LG_2015 | XQ62 | LG_2015/XQ62 | HLJ | A_B_{—} | S | S | Y |
| 2021P-16 | XQ62 | 2021P-16/XQ62 | JL | A_B_{—} | S | S | Y |
| JG528 | XQ62 | JG528/XQ62 | JL | A_B_{—} | S | S | Y |
| JG325 | XQ62 | JG325/XQ62 | JL | A_bb | W | W | Y |
| P-89 | XQ62 | P-89/XQ62 | JL | A_bb | W | W | Y |
| P-52 | XQ62 | P-52/XQ62 | JL | A_bb | W | W | Y |
| P-88 | XQ62 | P-88/XQ62 | JL | A_B_{—} | S | S | Y |
| JG511 | XQ62 | JG511/XQ62 | JL | A_B_{—} | S | S | Y |
| JG809 | XQ62 | JG809/XQ62 | JL | A_B_{—} | S | S | Y |
| JG856 | XQ62 | JG856/XQ62 | JL | A_B_{—} | S | S | Y |
| JH6 | XQ62 | JH6/XQ62 | JL | A_B_{—} | S | S | Y |
| TY8802 | XQ62 | TY8802/XQ62 | JL | A_B_{—} | S | S | Y |
| JG830 | XQ62 | JG830/XQ62 | JL | A_B_{—} | S | S | Y |
| JG576 | XQ62 | JG576/XQ62 | JL | A_B_{—} | S | S | Y |
| ZKF5 | XQ62 | ZKF5/XQ62 | JL | A_B_{—} | S | S | Y |
| DHX | XQ62 | DHX/XQ62 | JL | A_B_{—} | S | S | Y |
| JG81 | XQ62 | JG81/XQ62 | JL | A_B_{—} | S | S | Y |
| TH869 | XQ62 | TH869/XQ62 | JL | A_B_{—} | S | S | Y |
| GLYG | XQ62 | GLYG/XQ62 | JL | A_B_{—} | S | S | Y |
| YU | XQ62 | YU/XQ62 | JL | A_B_{—} | S | S | Y |

^{a} HLJ = Heilongjiang, and JL = Jilin.
^{b} S = Strong, and W = Weak.
^{c} Y = Yes, and N = Not.

### 1.5 Molecular Marker Development and Primer Design

With the NCBI website (https://www.ncbi.nlm.nih.gov/) and RFGB website (https://www.rmbreeding.cn), primers were designed within 100 bp around the candidate locus using IRGSPv1.0 as the reference genome.

### 1.6 DNA Extraction and Polyacrylamide Gel Electrophoresis

Referring to the DNA extraction method of Temnykh et al. (2001), the genomic DNA was extracted from 226 RILs derived from the cross of TH886/XQ62 and 120 breeding materials. The genomic DNA was used as a polymerase chain reaction (PCR) template. The products of the PCR reaction were assayed by polyacrylamide gel electrophoresis, then photographed with a gel imaging system after staining with ethidium bromide. Referring to the genotypes of parents, the progeny genotypes were identified.

### 2. Results

### 2.1 QTL Mapping

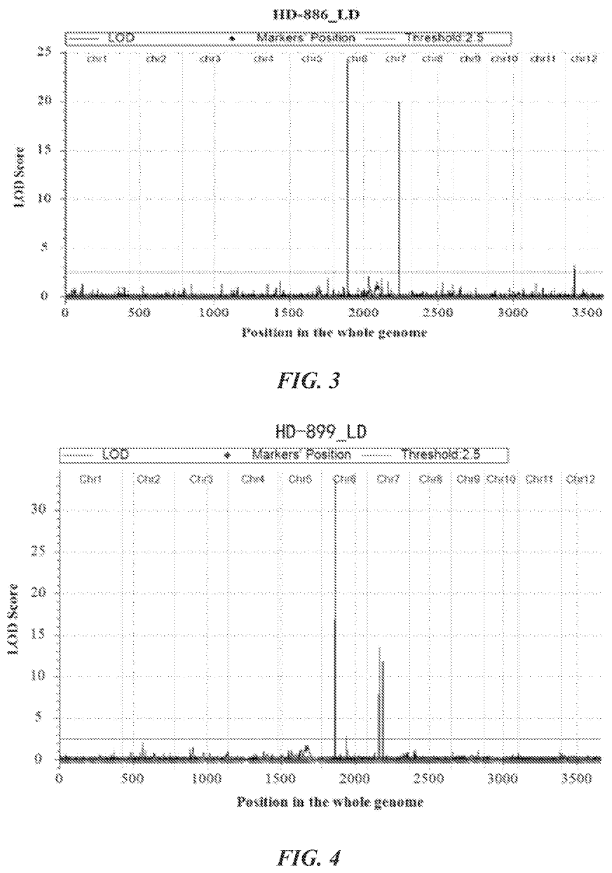

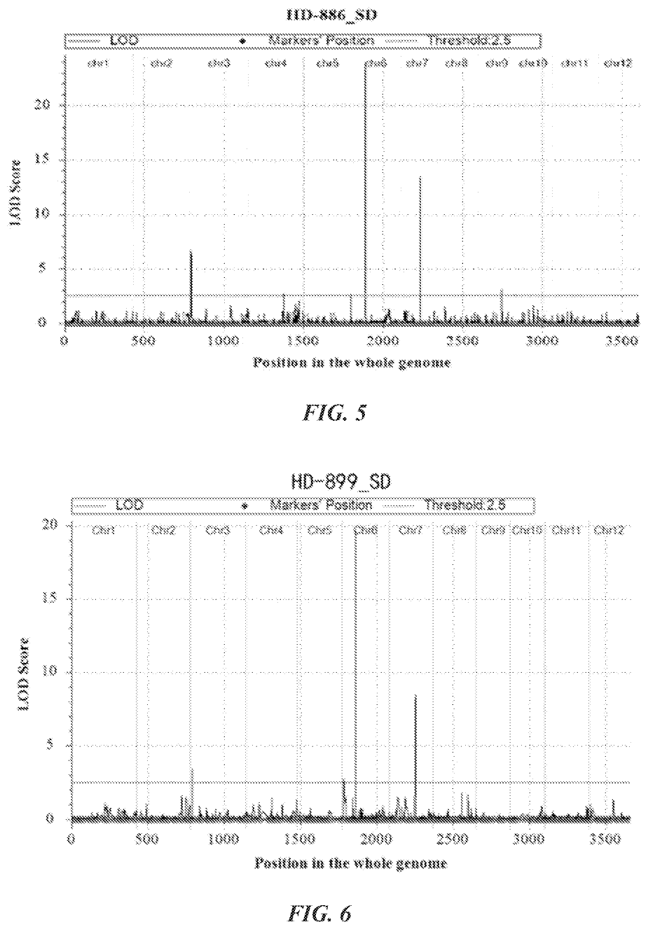

Using the two RIL populations TH886/XQ62 and TH899/XQ62, QTLs with high LOD values were located in the 8,665,233-9,600,319 bp interval on chromosome 6 and 8,556,052-11,072,552 bp interval on chromosome 7 under long-day conditions (FIG. 3 and FIG. 4), which were named qHd6 and qHd7 respectively. These two loci were also detected under SD condition (FIG. 5 and FIG. 6). Therefore, qHd6 and qHd7 are regarded as important candidate loci.

A molecular marker named ZLM7-1 was designed within 100 bp around the candidate gene of qHd7 using IRGSPv1.0 as the reference genome. The forward primer sequence is TCCCCCAAACATTTTCAGAACAC (SEQ ID NO: 1) and the reverse primer sequence is TAGGTGCAGTTGCAGTAGGT (SEQ ID NO: 2).

A known marker named M80410 (Deng Y, Zhai K, Xie Z, Yang D, Zhu X, Liu J, Wang X, Qin P, Yang Y, Zhang G, Li Q, Zhang J, Wu S, Milazzo J, Mao B, Wang E, Xie H, Tharreau D, He Z (2017) Epigenetic regulation of antagonistic receptors confers rice blast resistance with yield balance. Science 355 (6328): 962-965. doi:10.1126/science.aai8898) was found by this invention to be linked to the qHd6 locus for the photo-sensitivity trait. The forward primer sequence is GGATTGTCTTGTCTCTCTCGC (SEQ ID NO: 3), and the reverse primer sequence is CAGGACTTAGGGTTTCTCTCTTT (SEQ ID NO: 4). It can be used to identify the existence of Pigm gene and qHd6 locus, simultaneously.

### 2.2 Testing Cross and Marker Verification

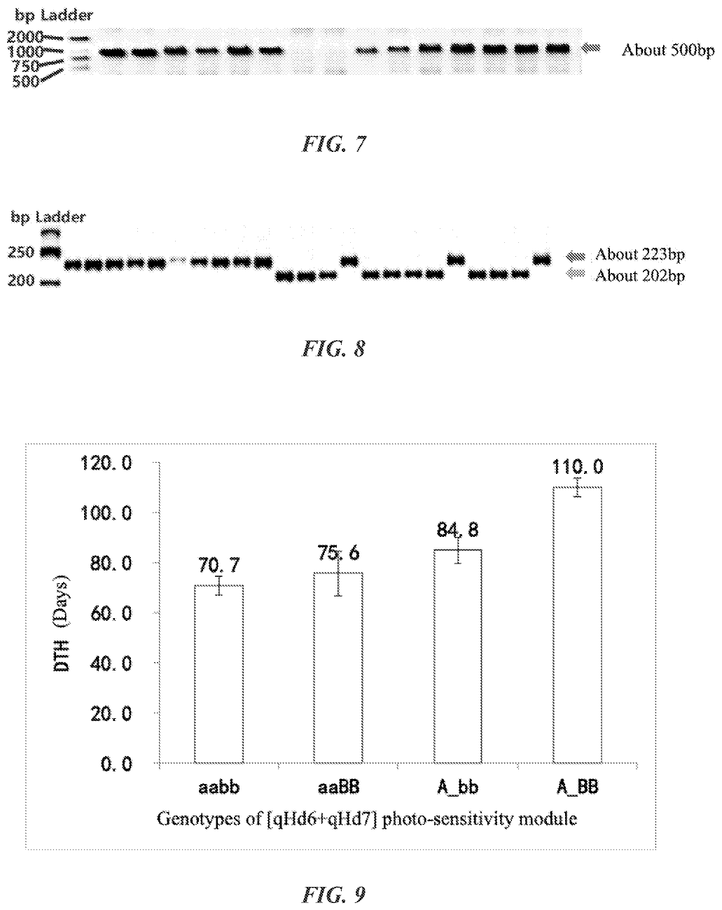

Using the developed molecular marker set [M80410+ZLM7-1] for the photo-sensitivity module, [qHd6+qHd7], the marker genotype selection was carried out in F₆ generation of RIL population derived from the cross TH886/XQ62. When the M80410 marker primer is used to amplify the rice genome template, the PCR amplification product is subjected to agarose gel electrophoresis, and the band type of PCR product is detected. If about 500 bp bands are amplified, the genotype of qHd6 site is detected as A_, otherwise, it is aa (FIG. 7). When ZLM7-1 marker primer is used to amplify rice genome template, the PCR amplification product is subjected to polyacrylamide gel electrophoresis. Because the amplification product of ZLM7-1 marker primer corresponding to the AA genotype at the qHd7 site is 21 bp more than that of ZLM7-1 marker primer corresponding to aa genotype, therefore, if about 223 bp bands are amplified, the qHd7 genotype is detected as BB; if about 202 bp bands are amplified, the qHd7 genotype is detected as bb (FIG. 8). The molecular marker set [M80410+ZLM7-1] is used in combination. Represented by progenies from a TPS cross, TH886/XQ62, when the marker genotype of the photo-sensitivity module, [qHd6+qHd7], is A_B_, strong TPS would appear. While the TPS of other genotypes are relatively weak (FIG. 9).

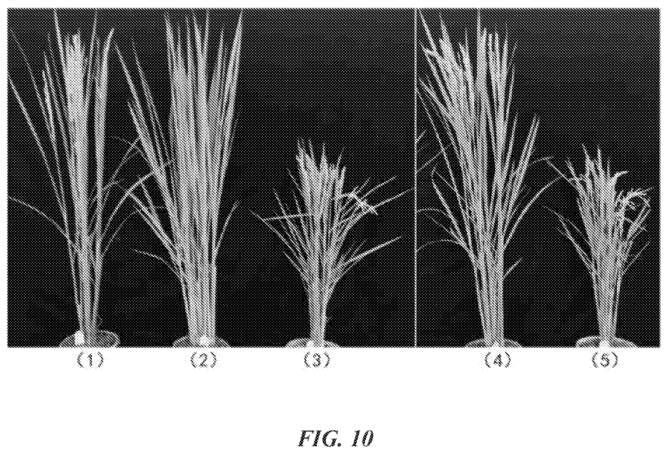

This invention also carries out the TPS prediction in the early Geng/japonica rice breeding materials from Heilongjiang and Jilin with the molecular marker set, [M80410+ZLM7-1], for the photo-sensitivity module [qHd6+qHd7]. A total of 35 breeding materials, including 19 from Jilin and 16 from Heilongjiang (Table 1), were randomly picked from 120 breeding materials and crossed with two Pigm near-isogenic lines, XQ62 and Kongyu 131 respectively. Among the F₁ derived from 19 Jilin materials, 16 (accounting for 84.2%) F₁ derived from crosses with XQ62 were found to pocess strong TPS, while no strong TPS was found the corresponding F₁ derived from crosses with Kongyu 131 (FIG. 10). Among the F₁ derived from 16 Heilongjiang materials, 7 (accouting for 43.8%) F₁ derived from crosses with XQ62 were found to pocess strong TPS, while no strong TPS was found the corresponding F₁ derived from crosses with Kongyu 131.

Using the molecular marker [M80410+ZLM7-1] of the [qHd6+qHd7] photosensitive module to carry out the marker genotype joint detection, the data showed that the predicted F₁ photo-sensitivity of the marker genotype was 88.6% consistent with the actual observation, and the predicted F₁ photo-sensitivity could also reach 75.0% for the breeding material with the female parent from Heilongjiang, and even 100% for the breeding material with the female parent from Jilin (Table 1).

In conclusion, it is effective to use the molecular marker [M80410+ZLM7-1] of the [qHd6+qHd7] photosensitive module to molecular marker assisted selection. especially in the photosensitive selection of the progeny of rice blast resistance improvement with the participation of the early Geng/japonica rice Pigm gene, and the selection of the resistant biological breeding parents of early japonica rice.

The above application does not limit the invention in any form.

1. A marker set for the photo-sensitivity module, [qHd6+qHd7], which is characterized by following points: one locus named qHd7 is located in 8,556,052-11,072,552 bp interval on chromosome 7 based on the reference genome of IRGSP v1.0;

2. A marker set for a photo-sensitivity module, which is characterized by following points: a combination of two loci, named qHd6 and qHd7, which are located in 8, 665,233-9,600,319 bp interval on chromosome 6 and 8,556,052-11,072,552 bp interval on chromosome 7, respectively, based on the reference genome of IRGSP v1.0 for rice.

3. The marker set for a photo-sensitivity module according to claim 2, which is characterized in that the marker set can be identified by using Polymerase Chain Reaction (PCR) with primers.

4. The marker set for a photo-sensitivity module according to claim 3, which is characterized by a PCR primer named M80410 for qHd6, with a forward primer sequence: GGATTGTCTTGTCTCTCTCGC (SEQ ID NO: 3), and a reverse primer sequence: CAGGACTTAGGGTTTCTCTCTTT (SEQ ID NO: 4); and by a PCR primer named ZLM7-1 for qHd7, with a forward primer sequence: TCCCCCAAACATTTTCAGAACAC (SEQ ID NO: 1), and a reverse primer sequence: TAGGTGCAGTTGCAGTAGGT (SEQ ID NO: 2).

5. A method for predicting the degree of transgressive photo-sensitivity (TPS) in northern early Geng/japonica rice progenies in breeding schemes using Pigm donor as parent according to claim 1, which is characterized by following genotyping method: the marker genotype of qHd6 can be identified as A_ or aa; the marker genotype of qHd7 can be identified as BB, bb or Bb; taking together, when the genotype of the photo-sensitivity module, [qHd6+qHd7], was identified as A_B_, the TPS of the northern early Geng/japonica rice with this genotype would be the strongest, and the TPS of the northern early Geng/japonica rice with other genotypes would be relatively weak.

6. The method according to claim 5, which is characterized by the following genotyping method: if a band of about 500 bp in size was amplified by the PCR primer M80410, the detection genotype for qHd6 would be A_; if a band of about 223 bp in size was amplified by the PCR primer ZLM7-1, the genotype for qHd7 would be BB; if a band of about 202 bp in size was amplified by the PCR primer ZLM7-1, the genotype for qHd7 would be bb. Taking together, if marker genotype of [M80410+ZLM7-1] for the photo-sensitivity module, [qHd6+qHd7], was identified as A_B_ in the northern early Geng/japonica progenies, there would appear strong TPS.

7. The method according to claim 6, which is characterized in that agarose gel electrophoresis or polyacrylamide gel electrophoresis is used to detect the bands, preferably agarose gel electrophoresis is used for the PCR amplification products of M80410 primers, and polyacrylamide gel electrophoresis is used for the PCR amplification products of ZLM7-1.

8. An application of molecular markers for blast-resistance related photo-sensitivity module, [qHd6+qHd7], in conducting parental selection or screening progenies carrying Pigm without strong TPS in blast-resistant biotechnology breeding schemes according to claim 1, the rice is northern early Geng/japonica derived from crosses with Pigm donor as one parent.

9. The application according to claim 8, which is characterized by using the molecular marker set [M80410+ZLM7-1] for the photo-sensitivity module [qHd6+qHd7], when the genotype of the photo-sensitivity module, [qHd6+qHd7], in the progenies of the northern early Geng/japonica were A_B_, strongest TPS would be predicted, while the TPS of other genotypes are relatively weak, the molecular marker set [M80410+ZLM7-1] can be used in parental selection or progenies screening carrying Pigm without strong TPS in blast-resistant biotechnology breeding schemes for early Geng/japonica rice.


SEQUENCE LISTING (4 sequences)

SEQ ID NO: 1 - DNA, 23 bp, Synthetic construct
  source 1..23: mol_type = other DNA
tcccccaaacattttcagaacac

SEQ ID NO: 2 - DNA, 20 bp, Synthetic construct
  source 1..20: mol_type = other DNA
taggtgcagttgcagtaggt

SEQ ID NO: 3 - DNA, 21 bp, Synthetic construct
  source 1..21: mol_type = other DNA
ggattgtcttgtctctctcgc

SEQ ID NO: 4 - DNA, 23 bp, Synthetic construct
  source 1..23: mol_type = other DNA
caggacttagggtttctctcttt